(19) United States
(12) Patent Application Publication
(10) Pub. No.: US 2020/0141928 A1
(43) Pub. Date: May 7, 2020

(54) METHOD FOR RNA TAGGING AND ANALYSIS ON SINGLE CELL

(71) Applicant: Centro Cardiologico Monzino S.P.A.

(72) Inventors: Marina Camera, Milano (IT); Chiara Zara, Milano (IT); Elena Tremoli, Milano (IT)

(73) Assignees: Centro Cardiologico Monzino S.P.A., Milano (IT); Centro Cardiologico Monzino S.P.A., Milano (IT)

(21) Appl. No.: 16/305,071

(22) PCT Filed: Jun. 22, 2017
(86) PCT No.: PCT/IB2017/053733

§ 371 Date: Nov. 28, 2018

(30) Foreign Application Priority Data
Jun. 23, 2016 (IT) 102016000064945

(51) Int. Cl. G01N 33/50; C12N 5/078; G01N 33/58; C12Q 1/68

(52) U.S. Cl. CPC G01N 33/5094; C12Q 1/68; G01N 33/58; C12N 5/0644

(57) ABSTRACT

The present invention relates to a method for RNA tagging and analysis on single cell, suitable for platelets and cells with a short half-life, comprising the following steps: a) Providing a population of cells of interest; b) Incubating said cells for a time of 10 minutes to 3 hours, or 30 minutes to 2 hours, at a temperature from about 20° C. to about 37° C., in a culture medium supplemented with a lipofection reagent and a SmartFlare™ probe of interest; c) Fixing with a fixative; d) Visualizing and analysing the RNA of interest.

The present invention relates to a method for RNA tagging and analysis on single cell, suitable for platelets and cells with a short half-life, comprising the following steps:
a) Providing a population of platelets and/or cells of interest;
b) Incubating said platelets and/or cells for a time of 10 minutes to 3 hours, or 30 minutes to 2 hours, at a temperature from about 20° C. to about 37° C., in a culture medium supplemented with a lipofection reagent and a SmartFlare™ probe of interest;
c) Fixing with a fixative;
d) Visualizing and analysing the RNA of interest.

### BACKGROUND ART

Platelets are corpuscolar blood elements playing a crucial role in coagulation. The crucial role played by platelets in coagulation makes them key players in thrombotic phenomena. Recently, other functions have been attributed to platelets, such as, vascular integrity control, involvement in inflammatory and immune processes, tumour metastasis, angiogenesis and, last but not least, the onset and progression of atherothrombotic disease. The characteristic which distinguishes platelets is that they are core-free. Conversely, platelets possess granules, cytoplasmic organelles, and RNA.

Given the multifaceted role played by platelets and the presence of RNA therein, the need for tagging and analyzing the RNA contained therein is strongly felt.

The SmartFlare™ RNA detection probe technology (Merck Millipore, Germany) allows for the analysis of intracellular RNA expression on single living cell. The SmartFlare™ method includes plating the cells, preferably in 96-well plates, at a 60-80% confluence. The SmartFlare™ probe is added to the culture and allowed to incubate for a period of about 16 hours, so as to allow it to enter the cells via endocytosis. Once inside the cell, the probe, recognizing a specific target, binds thereto and a subsequent fluorescence analysis reveals the presence thereof. Since platelets, as well as other cells with short half-life, such as monocytes, lymphocytes, granulocytes, may not be maintained in culture for a time as long as that required by the method, they are not suitable for the analysis by means of the above method.

The first attempts to introduce nucleic acids into platelets have been described by Hong W. et al. Transfection of Human Platelets with Short Interfering RNA, Clin Transl Sci. 2011; 4 (3): 180-182, though achieving a very low efficiency, equal to about 9%. Method improvements are described in WO 2014118817, with the purpose of using siRNA in platelets. However, no attempt is described about platelet RNA tagging, in particular, mRNA and miRNA, which tagging is thus not readily obtainable with the technologies available to date. In particular, a strong need for tagging RNA species modulated in acute pathology conditions is felt. Therefore, a method which allows to tag platelet RNA and cells with short half-life in vitro is needed, in order to overcome the limits imposed by the characteristics of the platelets themselves.

### DESCRIPTION OF THE INVENTION

The present invention describes a method which surprisingly allows to overcome the limits imposed by the particular nature of platelets and cells with short half-life, thus leading to an efficient tagging of platelet RNA and cells with short half-life in vitro.

### DESCRIPTION OF THE DRAWINGS

FIG. 1: platelet integrity analysis performed at the cytofluorimeter after treatment according to the Smartflare™ method (comparative).

FIG. 2: platelet integrity analysis performed at the cytofluorimeter after treatment according to the method of the present invention.

FIG. 3: comparative analysis of different lipofection reagents for the incorporation of the Smartflare™ probe, negative control (unflare) and positive control (uptake) in platelets.

FIG. 4: Exemplary mRNA (18S and TF) expression levels obtained according to the method of the present invention.

### DETAILED DESCRIPTION OF THE INVENTION

It is herein described an innovative method for RNA tagging and analysis on single cell, suitable for platelets and cells with a short half-life, comprising the following steps:
a) Providing a population of platelets and/or cells of interest;
b) Incubating said platelets and/or cells for a time of 10 minutes to 3 hours, or 30 minutes to 2 hours, at a temperature from about 20° C. to about 37° C., in a culture medium supplemented with a lipofection reagent and a SmartFlare™ probe of interest;
c) Fixing with a fixative;
d) Visualizing and analysing the RNA of interest.

In a preferred embodiment, said RNA is mRNA or miRNA.

For the aim of the present invention, SmartFlare™ probes mean probes provided by Merck Millipore, which recognize mRNA or miRNA key target. Said probes are Cy5 or Cy3 tagged. However, by means of the methods known to those skilled in the art, further probes may be synthesised for specific purposes, i.e. probes recognizing targets of particular interest. For the aim of the present invention, SmartFlare™ probes are probes capable of entering a cell, recognizing a target RNA of interest, and making it detectable by the SmartFlare™ method; by way of not limiting example, the SmartFlare™ probes found in the Merck Millipore catalogue are included. Also included are those additional probes which may be synthesised by those skilled in the art for further targets and purposes of interest, in particular oligonucleotide sequences capable of pairing with target RNAs, such as by way of example DNA sequences, tagged with a fluorophore detectable by cytofluorimetry and microscopy.

In a preferred embodiment, said method is operated on platelets. Preferably, said incubation takes place for about 1 hour at room temperature, or at about 37° C. Preferably, said culture medium is RPMI1640 supplemented with glutamine or GlutaMAX™.

In a still more preferred embodiment, about 500,000 platelets resuspended in about 100 μl of culture medium are provided. Preferably, said lipofection reagent is selected from: TransIT-LT1 (Mirus), Turbofect transfection reagent (Invitrogen), RiboJuice™ siRNA Transfection Reagent (Merck Millipore), NanoJuice® Transfection Kit (Merck Millipore), GeneJuice® Transfection Reagent (Merck Millipore) and other transfecting reagents known to those skilled in the art. The experimental data obtained and reported below showed that, despite the great issues, well known in the prior art, relating to the introduction of nucleic acids in platelets, it was surprisingly possible to achieve more than satisfactory transfection levels using TransIT-LT1 (Mirus), Gene Juice, Nano Juice, Ribo Juice (Merck Millipore) and Turbofect transfection reagent (Invitrogen). From a morphological analysis, it has been observed that, upon transfection, the platelets do not exhibit major alterations.

1.5 μl of said lipofection reagents, 1:10 diluted, are added to 100 μl of the culture medium. Preferably, said culture medium contains 300 pM of said SmartFlare™ probe.

Preferably, said fixative used in said step c) is 1% paraformaldehyde (PFA).

Optionally, at the end of the incubation as described in step b) and before said fixing step c), said platelets are tagged with a specific antibody, for example FITC-tagged antiCD41. Thereby, platelets containing the RNA of interest may be detected.

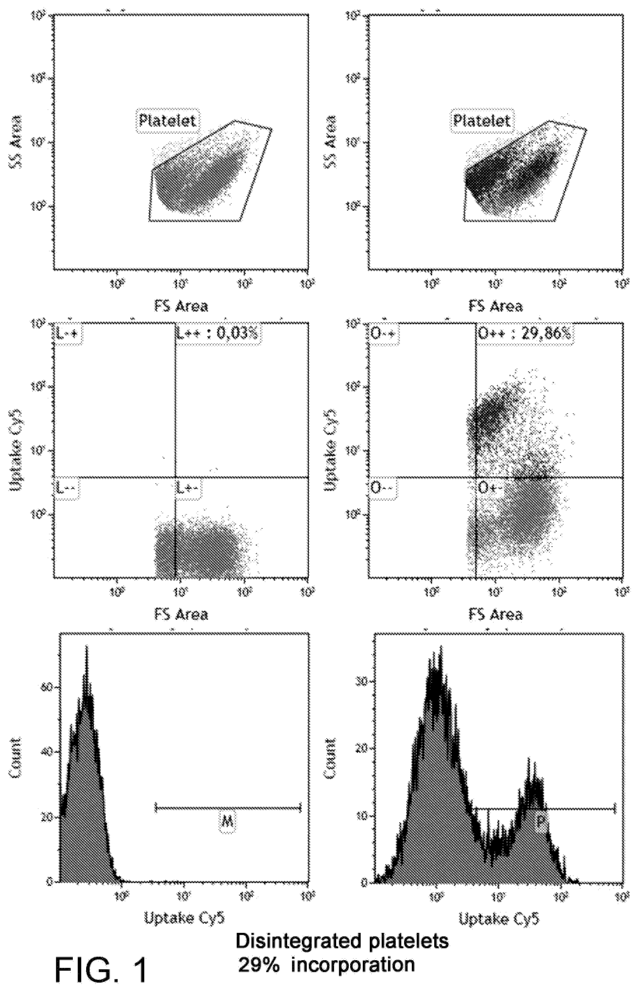

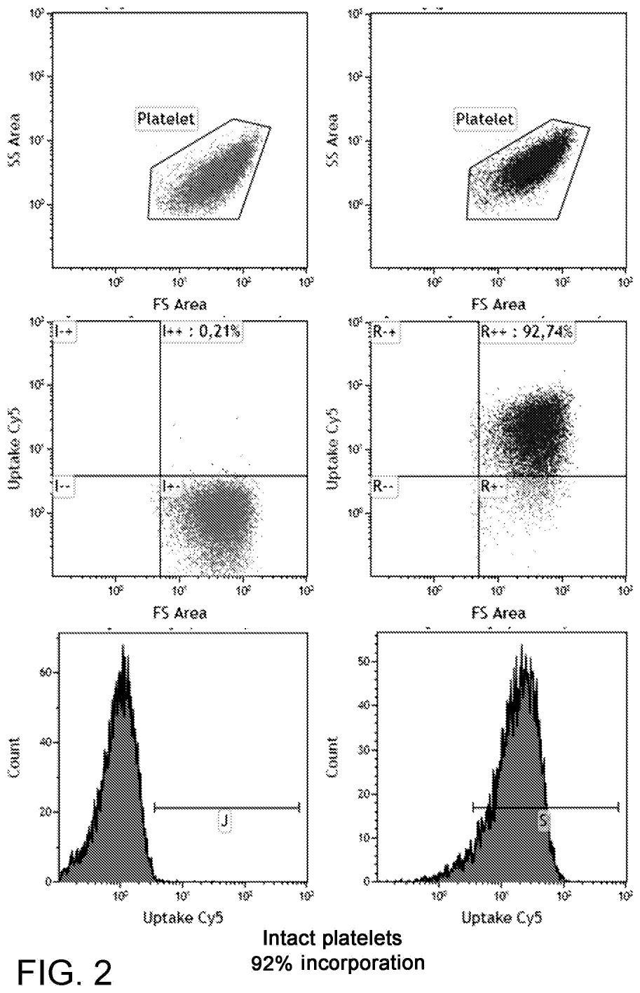

FIGS. 1 and 2 show platelet integrity data using the method as proposed by SmartFlare™ technology and the method according to the present invention, respectively. It is apparent that a marked morphological platelet alteration occurs by applying the method known from the prior art, thus making the method not applicable to the platelets, and that the method described herein surprisingly obviates such a morphological alteration and keeps the platelets intact and therefore sensitive to tagging.

It is particularly efficient, for the purposes of the present invention, isolating platelets from whole blood by the method described below. The blood is collected by venous withdrawal from the cephalic vein of donors who have signed their informed consent to participate in the study. Whole blood (WB) was taken by means of a 19-gauge needle, without venous stasis, and placed in a tube containing citrate ( 1/10 of 0.129 M sodium citrate volume) and corn trypsin inhibitor (50 μg/ml) (Vacutainer, Becton Dickinson) discarding the first 4 ml. For platelet preparation, WB was centrifuged at room temperature, in the absence of a brake, preferably at 100 g for 15′. For the aim of the present invention, the total platelet preparation analysed by the Sysmex XE-2100 Automated Hematology Analyzer is used to determine the platelet recovery, the platelet average volume (MPV), the platelet immature fraction (IPF), and the platelet distribution width (PDW).

Some examples of results obtained by the method according to the present invention are shown hereinbelow. Said examples are not intended to limit in any manner the scope of the present invention to that specifically exemplified herein. In particular, the results obtained using probes for the Tissue Factor (TF mRNAs) and 18S in platelets are herein described. However, probes for further RNAs of specific interest may be synthesised and used according to methods known to those skilled in the art.

### EXAMPLES

### Example 1

### Comparison of the Incorporation Efficiency of the SmartFlare™ Probe

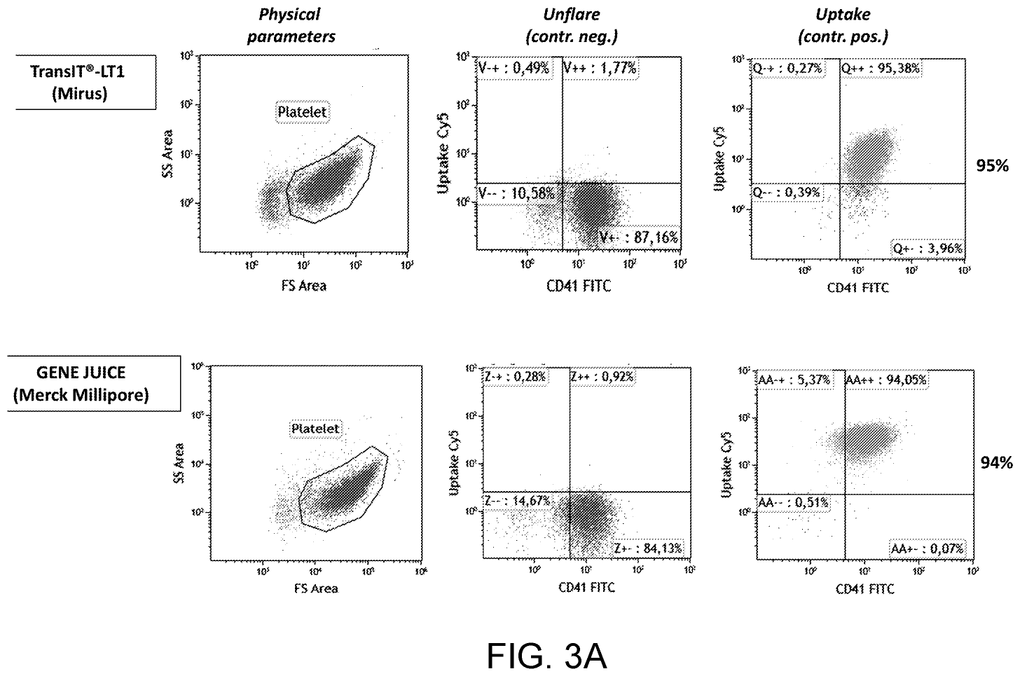

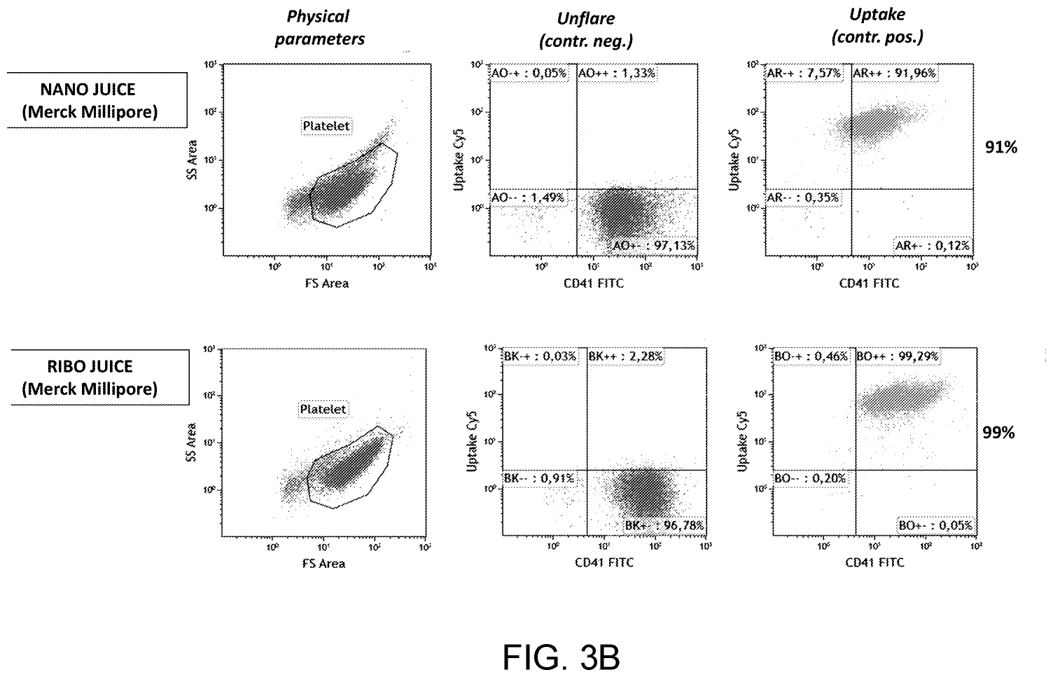

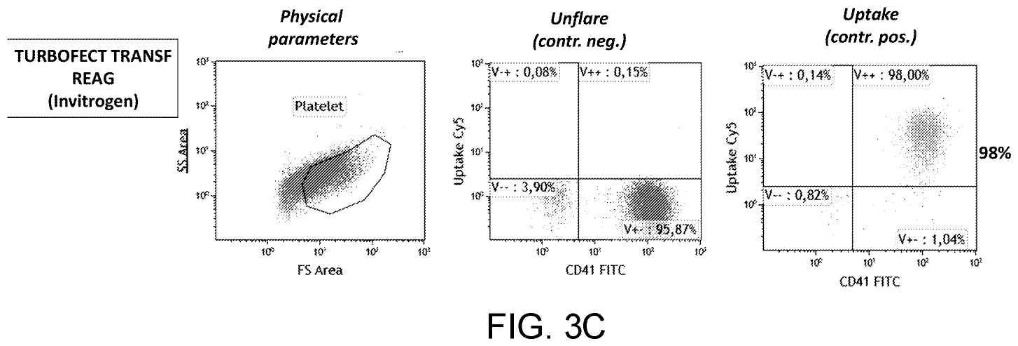

A platelet preparation was incubated with a SmartFlare™ probe for the uptake control. Each sample was incubated in the presence of one of the lipofection reagents shown in FIG. 3 and 300 pM of the Uptake Cy5 probe and the negative control (Scramble Cy5) for 1 hour at room temperature in RPMI1640 culture medium supplemented with glutamine. By means of the Kaluza image analysis software, the fluorescence degree was quantified and percent values of the platelets expressing the positive control signal were obtained, by subtracting the signal from the negative control in order to remove the unspecified signal.

The results are shown in FIG. 3. The reported data surprisingly show that the solution of the present invention can introduce nucleic acids in platelets, more specifically probes for detecting tagged RNA, thus surprisingly allowing a platelet RNA to be analysed on living single cell. It is worth noting the high efficiency obtained, as shown on the right in FIG. 3, with each of the lipofection reagents reported.

### Example 2

### Tagging and Analysis of TF and 18S mRNA in Platelets

It is known that a subpopulation of human platelets express TF mRNA. The used probes include:
Control probes: 18S-Hu-Cy5 Smartflare™ (Cat No. SF-142); negative control: Scramble-Cy5 SmartFlare™ (Cat No. SF-102) which binds non-sense mRNA sequences not present in the sample; positive control: Uptake-Cy5 SmartFlare™ (Cat No. SF-137) having a constitutively fluorescent fluorophore.
Specific probes were designed for mRNAs of interest, in particular a Cy5 tagged probe for TF mRNA, SEQ ID NO. 1 (GTTTCACACCTTACCTGGAGACAAACC).

The platelets were isolated from whole blood of healthy volunteers after signature of the informed consent, according to methods known to those skilled in the art.

For each of the above probes, 500,000 platelets were incubated for 1 hour at room temperature in RPMI1640 culture medium supplemented with glutamine with 1.5 μl of transfection reagent Transit-LT1 1:10 diluted and 300 pM of one of the above SmartFlare™ probes.

After said incubation, the platelets were tagged with a FITC-tagged anti-CD41 antibody and fixed with 1% PFA. By cytofluorimetry, the Cy5 fluorescence of the probes was analysed in the CD41 positive platelets.

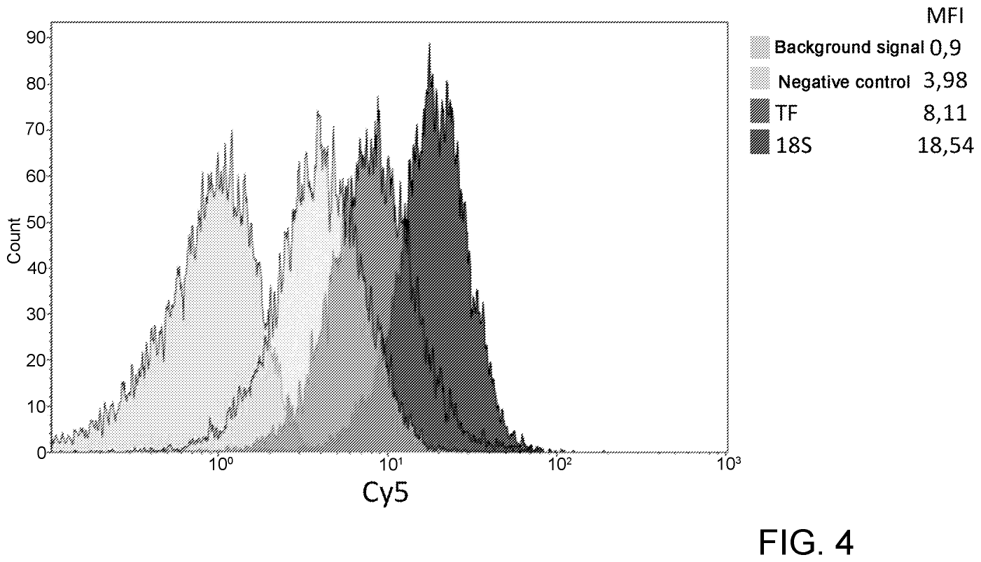

Once the platelet aggregates have been excluded, the signal from the positive control (Uptake Cy5) and the negative control (Scramble Cy5) was assessed. By means of the Kaluza analysis software, the median fluorescence intensity (MFI) of the probes which respectively detect 18S and TF mRNAs between the CD41 and Cy5 positive events was quantified, using Scramble Cy5 as the negative control to subtract the unspecified signal. The data are expressed as MFI and the results are shown in FIG. 4. In particular, TF was found to be expressed at low levels in the platelets versus 18S which is very abundant.

1. A method for RNA tagging and analysis on single cell, suitable for platelets and cells with a short half-life, comprising the following steps:
a) providing a population of cells of interest;
b) incubating said platelets and/or cells for a time of 10 minutes to 3 hours, at a temperature from about 20° C. to about 37° C., in a culture medium supplemented with a lipofection reagent and a probe capable of recognizing selected RNA inside single cell of interest;
c) fixing with fixative;
d) visualizing and analysing the RNA of interest.

2. The method according to claim 1, wherein said cell population is a population of human platelets.

3. The method according to claim 1, wherein said RNA is mRNA and miRNA.

4. The method according to claim 1, wherein said incubation occurs for about 1 hour at room temperature in RPMI1640 culture medium.

5. The method according to claim 1, wherein approximately 500,00 platelets resuspended in about 100 μl of culture are provided.

6. The method according to claim 1, wherein 1.5 μl of lipofection reagent diluted 1:10 is added to 100 μl of said culture medium contacting 300 pM of said probe.

7. The method according to claim 2, wherein said platelets are tagged with a platelet-specific antibody at the end of the incubation of step b) and before said fixing step c).
